(19) United States
(12) Patent Application Publication
(10) Pub. No.: US 2021/0015428 A1
(43) Pub. Date: Jan. 21, 2021

(54) SAMPLING CAPSULE AND SAMPLING CAPSULE SYSTEM

(71) Applicant: ANKON TECHNOLOGIES CO., LTD, Wuhan (CN)

(72) Inventors: Tianyi Yangdai, Wuhan (CN); Yuhui BAO, WUHAN (CN); Hangyu PENG, WUHAN (CN); Fanhua MING, Wuhan (CN)

(73) Assignee: ANKON TECHNOLOGIES CO., LTD, Wuhan (CN)

(21) Appl. No.: 16/930,056

(22) Filed: Jul. 15, 2020

(30) Foreign Application Priority Data
Jul. 15, 2019 (CN) 201910636318.1

(51) Int. Cl. A61B 5/00; A61B 10/00; A61B 1/04; A61B 5/07; A61B 5/145; A61B 8/12

(52) U.S. Cl. CPC A61B 5/6861; A61B 10/0045; A61B 1/041; A61B 5/073; A61B 5/14539; A61B 2562/06; A61B 8/12; A61B 2010/0061; A61B 2562/162; A61B 2560/0475; A61B 5/14503

(57) ABSTRACT

A sampling capsule and sampling capsule system are provided. The sampling capsule includes an enclosure, a sampling assembly, a mucosal flora collection auxiliary assembly and a control module. The sampling assembly includes a sample chamber disposed in the enclosure, an outer sampling port on the enclosure, a connecting tube connecting the outer sampling port and the sample chamber, and a sampling switch for opening or closing the connecting tube. The mucosal flora collection auxiliary assembly includes a vibration motor disposed in the enclosure, and/or a counterweight at the outer sampling port. The control module includes a microprocessor in communication with the sampling switch and the vibration motor.

### CROSS-REFERENCE OF RELATED APPLICATIONS

The application claims priority to Chinese Patent Application No. 201910636318.1 filed on Jul. 15, 2019, the contents of which are incorporated by reference herein.

### FIELD OF INVENTION

The present invention relates to a medical device, and more particularly to a sampling capsule and sampling capsule system for the collection of mucosal flora in gastrointestinal tract.

### BACKGROUND

Sampling capsule is an intelligent capsule introduced into gastrointestinal tract to sample digestive fluids at various sites. Taking the sampling of digestive fluids in intestinal tract as an example, intestinal microbiota are closely related to human health. Medical research has found that more and more diseases are related to intestinal microbiota, such as: cardiovascular disease, obesity, diabetes, various intestinal diseases (IBD, IBS, CD, SIBO, etc.), liver disease, allergies, immune disease, neurological diseases (autism, depression, Alzheimer's), cancer, hypertension, chronic kidney disease, etc. To study the pathology of these diseases in depth, the study of intestinal flora, especially the distribution and abundance of flora in the large and small intestine, has received increasing attention.

There are significant differences in the type and abundance of flora in large and small intestines, so they need to be studied separately. In addition, due to the structure of gastrointestinal tract and the characteristics of colony reproduction therein, the intestinal flora can be divided into mucosal flora (or flora on mucosa) and luminal flora (or flora in intestinal lumen). The luminal flora is more mobile, and thus there are differences in abundance, distribution, and species compared to the mucosal flora of the same site. The colonies of mucosal flora colonize mostly in the mucus layer of the mucosa, so the abundance of mucosal flora is relatively high. The study of mucosal flora is of greater value.

Currently, the more traditional methods of intestinal flora analysis include: suction by enteroscope, mucosal collection by enteroscope, stool analysis, and hydrogen breath test. However, these methods either require the use of an enteroscope, which brings much more pain to the subject, or can only analyze the flora in the posterior colon and rectum, with fewer means of studying the mucosal flora of the small intestine, especially the terminal ileum.

Capsule endoscopy-based intestinal microflora collection technique has advantages such as comfort and full coverage of the gastrointestinal tract, thus it can be an effective tool for research in this field. Currently, a plurality of collection systems based on capsule endoscopy have been designed. According to the nature of the samples collected, the systems can be broadly categorized into: tissue sampling systems and fluid sampling systems.

Tissue sampling systems are large and complex. During biopsy, a collection device, such as a blade, biopsy forceps, etc., needs to be extended from the capsule endoscopy. For details, refer to “The research of a biopsy mechanism for capsule robot”, “A rotational micro biopsy device for the capsule endoscope”, “A novel microactuator for microbiopsy in capsular endoscopes”, “Shape memory alloy based biopsy device for active locomotive intestinal capsule endoscope”, “Magnetic torsion spring mechanism for a wireless biopsy capsule”, “Design of Micro Biopsy Device for Wireless Autonomous Endoscope”, etc. Since the control and positioning capabilities of capsule endoscopy are significantly weaker than traditional endoscopes, and it is hard to immobilize the capsule during biopsy and impossible to stop bleeding after the procedure, tissue sampling systems may pose a certain safety risk.

Fluid sampling systems usually absorb fluids from the gastrointestinal tract by some kind of power, such as adsorption of water absorbing material, suction of a micro-pump, vacuum suction, etc., and analyze the flora in the fluids after recovery. For details, refer to “Ingestible Gastrointestinal Sampling Devices: State-of-the-Art and Future Directions”, “The study of a remote-controlled gastrointestinal drug delivery and sampling system” and other studies. Such systems come with high safety and collection success rate. However, the systems typically only absorb fluids from the lumen and analyze primarily the luminal flora, with no effective method for mucosal flora collection.

It is necessary to provide an improved sampling capsule and sampling capsule system to solve the said problem.

### SUMMARY OF THE INVENTION

The present invention aims to provide a sampling capsule and sampling capsule system for the collection of mucosal flora in gastrointestinal tract.

In order to achieve the object, the following technical solutions are employed.

The present invention provides a sampling capsule, comprising:

an enclosure; a sampling assembly comprising a sample chamber disposed in the enclosure, an outer sampling port on the enclosure, a connecting tube connecting the outer sampling port and the sample chamber, and a sampling switch for opening or closing the connecting tube;

a mucosal flora collection auxiliary assembly comprising a vibration motor disposed in the enclosure, and/or a counterweight at the outer sampling port; and

a control module comprising a microprocessor in communication with the sampling switch and the vibration motor.

In one embodiment, the sampling assembly comprises a plurality of outer sampling ports and a sampling cavity connected to the plurality of outer sampling ports, and the connecting tube is connected to the sampling cavity.

In one embodiment, the plurality of outer sampling ports are distributed with certain spacing along the circumference of the enclosure.

In one embodiment, the aperture diameter of each of the outer sampling ports is smaller than the inner diameter of the connecting tube.

In one embodiment, the sampling cavity comprises a filtering structure.

In one embodiment, the vibration motor is a button-type vibration motor, a coreless motor with an eccentric device, or a linear vibration motor.

In one embodiment, the vibration motor is disposed in the central position of the sampling capsule.

In one embodiment, the counterweight is a magnetic member and the center of gravity of the magnetic member is inclined to a side of the magnetic member close to the enclosure.

In one embodiment, one side of the magnetic member is curved to match the enclosure, and the magnetic member is radially magnetized.

In one embodiment, the magnetic member and the outer sampling port are arranged side-by-side along the axis of the sampling capsule.

In one embodiment, the sampling capsule further comprises a sample drawing assembly, the sample drawing assembly comprising a drawing port on the enclosure and connected to the sample chamber, a fixing member corresponding to the drawing port, and a silicone plug fitted in the fixing member.

In one embodiment, the control module further comprises a sensor for collecting physiological parameters and/or image information in gastrointestinal tract, and the sensor communicating with the microprocessor; or the control module further comprises a sensor for collecting physiological parameters and/or image information in the gastrointestinal tract, and a storage module for storing normal physiological parameters or image information and physiological parameters or image information in case of possible lesions in different regions of the gastrointestinal tract, wherein both the sensor and the storage module communicate with the microprocessor; or the control module further comprises a sensor for collecting physiological parameters and/or image information in the gastrointestinal tract and a wireless transmission module for communicating with an external device, wherein the sensor communicates with the microprocessor.

In one embodiment, the sensor is one or more sensors selected from an image sensor, a pH sensor, or an ultrasonic sensor, wherein when the sensor comprises an image sensor, part of the enclosure is transparent, and when the sensor comprises a pH sensor, the enclosure comprises a window.

The present invention further provides a sampling capsule system, comprising a sampling capsule and an external device, the external device is in communication with a control module of the sampling capsule; the sampling capsule comprising:

an enclosure;

a sampling assembly comprising a sample chamber disposed in the enclosure, an outer sampling port on the enclosure, a connecting tube connecting the outer sampling port and the sample chamber, and a sampling switch for opening or closing the connecting tube;

a mucosal flora collection auxiliary assembly comprising a vibration motor disposed in the enclosure, and/or a counterweight at the outer sampling port; and the control module comprising a microprocessor in communication with the sampling switch and the vibration motor.

Compared with the prior art, the present invention has the following beneficial effects: the sampling capsule of the present invention can assist the sampling assembly to collect mucosal flora through the mucosal flora collection auxiliary assembly, can suck sample fluid into the sample chamber by opening the connecting tube by the sampling switch, and after sampling is completed, can close the connecting tube by the sampling switch to prevent the sample from leaking or being contaminated, and can precisely control the sample volume by controlling the opening state and time of the connecting tube. In addition, the sampling switch actively controls the opening or closing of the connecting tube to make the sampling capsule free to suck sample fluid in any region of the gastrointestinal tract without being affected by a special environment therein. Therefore, the sampling capsule has a high versatility.

### BRIEF DESCRIPTION OF THE DRAWINGS

FIG. 1 is an axial sectional view of a sampling capsule according to a preferred embodiment of the present invention.

FIG. 2 is a cross sectional view of the sampling capsule of FIG. 1.

FIG. 3 is a structural view of a magnetic member of the sampling capsule of FIG. 1 according to a first embodiment of the present invention.

FIG. 4 is a structural view of a magnetic member of the sampling capsule of FIG. 1 according to a second embodiment of the present invention.

FIG. 5 is a structural view of a magnetic member of the sampling capsule of FIG. 1 according to a third embodiment of the present invention.

FIG. 6 is a structural view of a magnetic member of the sampling capsule of FIG. 1 according to a fourth embodiment of the present invention.

FIG. 7 is a structural view of a magnetic member of the sampling capsule of FIG. 1 according to a fifth embodiment of the present invention.

FIG. 8 is a schematic view showing the sampling capsule that is collecting mucosal flora.

FIG. 9 is a schematic view of the direction defined based on the sampling capsule.

### DETAILED DESCRIPTION

The present invention can be described in detail below with reference to the accompanying drawings and preferred embodiments. However, the embodiments are not intended to limit the invention, and the structural, method, or functional changes made by those skilled in the art in accordance with the embodiments are included in the scope of the present invention.

In the figures of the present invention, some sizes of a structure or portion may be exaggerated relative to other structures or portions for ease of illustration, and thus, are merely used to illustrate the basic structure of the subject matter of the present invention.

In addition, “and/or” as used herein denotes “or” or “and”, e.g., “M and/or N” comprises M, or N, or M and N.

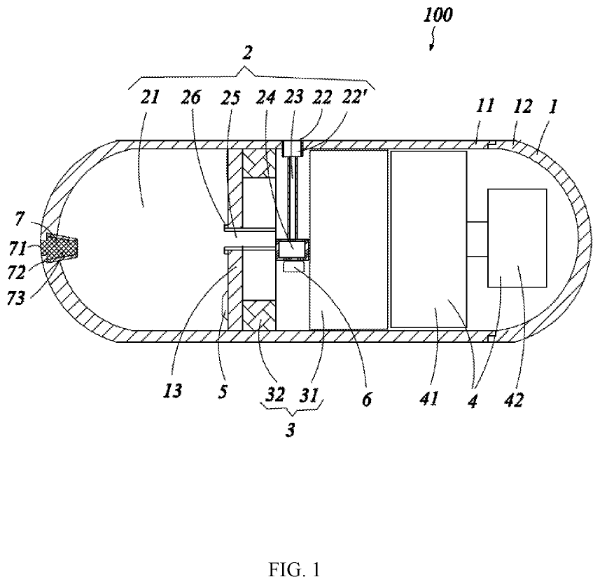

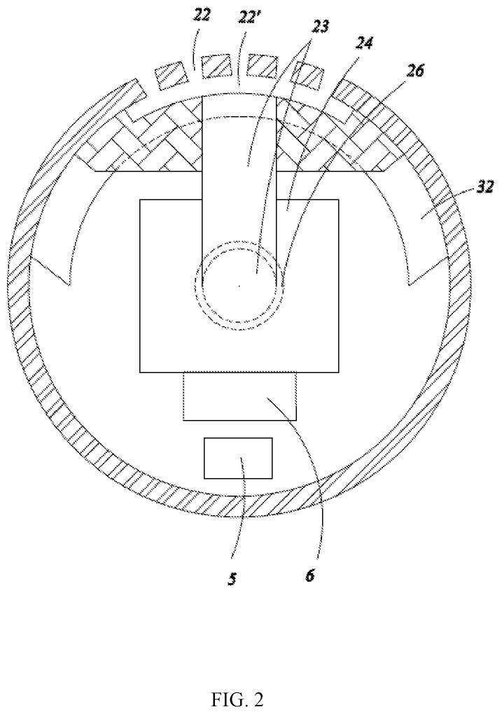

Referring to FIG. 1 to FIG. 2, a preferred embodiment of a sampling capsule 100 is shown according to the present invention. The sampling capsule 100 comprises an enclosure 1, a sampling assembly 2 for collecting mucosal flora, a mucosal flora collection auxiliary assembly 3 for assisting mucosal flora collection and a control module 4. The control module 4 comprises a microprocessor in communication with at least one or more structures of the assemblies to control and/or coordinate the working state thereof.

The enclosure 1 is biocompatible and cannot be corroded by digestive fluids, and can be set as transparent or opaque as needed. Moreover, the enclosure 1 is constructed by at least two parts joined together to facilitate arrangement of internal components. For example, as shown in FIG. 1, the enclosure 1 is composed of a first enclosure 11 and a second enclosure 12 which are distributed along the longitudinal direction of the sampling capsule 100, and are screwed or glued together.

The sampling assembly 2 comprises a sample chamber 21 disposed in the enclosure 1, an outer sampling port 22 on the enclosure 1, a connecting tube 23 connecting the outer sampling port 22 and the sample chamber 21, and a sampling switch 24 for opening or closing the connecting tube 23.

Specifically, the sampling capsule 100 further comprises a partition wall 13 arranged in the enclosure 1. The sample chamber 21 is enclosed by the partition wall 13 and the enclosure 1 on a first side of the partition wall 13. The outer sampling port 22 is on a second side of the partition wall 13. The sampling assembly 2 further comprises an inner sampling port 25 in the partition wall 13. The connecting tube 23 connects the outer sampling port 22 and the inner sampling port 25.

The partition wall 13 is designed integrally with the enclosure 1 on the first side of the partition wall 13 to form the sample chamber 21 with a good leak tightness. Or the partition wall 13 and the enclosure 1 on the first side of the partition wall 13 have a split-type design and the tightness at the junction of the two ensures that the sample chamber 21 can maintain its required vacuum.

Before use, the sample chamber 21 is sterilized, and the sample chamber 21 is in vacuum with an absolute pressure between 0 hPa and 260 hPa. Methods of evacuating the sample chamber 21 comprises, but are not limited to, opening the connecting tube 23 before completion of manufacturing, extracting air from the sample chamber 21 by a pumping device, and after achieving a desired vacuum, closing the connecting tube 23 so that the sample chamber 21 maintains the desired vacuum. Alternatively, before use, extracting air from the sample chamber 21 through a sample drawing assembly 7 by a pumping device to achieve the desired vacuum.

The connecting tube 23 is a flexible tube, preferably a silicone tube. The connecting tube 23 is connected to the partition wall 13 using a connecting piece 26 so that the connecting tube 23 is connected to the inner sampling port 25 with good leak tightness.

In one embodiment, the connecting piece 26 is a UV adhesive, which glues the connecting tube 23 to the perimeter of the inner sampling port 25 in the partition wall 13. In other embodiment, the connecting piece 26 is a rubber ring, which is put on the connecting tube 23 or fit into the inner sampling port 25, and after assembly the rubber ring arranges between the connecting tube 23 and the inner sampling port 25 under an interference fit for sealing and connecting.

In addition, the sampling switch 24 is disposed at a middle position of the connecting tube 23. The connecting tube 23 on either side of the sampling switch 24 may be one silicone tube or two split connecting tubes 23.

The sampling switch 24 can be a piezoelectric miniature valve, a shape memory metal miniature valve, or a gas tube clamp, etc. The piezoelectric miniature valve and the shape memory metal miniature valve control the opening and closing of the connecting tube 23 using prior art, which is not be repeated here, while the gas tube clamp can be designed with reference to the methods in Chinese Patent Applications No. 201811219936.8, 201811219926.4 and 201810617763.9 and is not be repeated here.

The sampling switch 24 is normally in a closed state, maintaining the vacuum in the sample chamber 21 and the connecting tube 23 connecting the sample chamber 21 and the sampling switch 24, so that it can withstand a pressure of at least one atmosphere. When the microprocessor receives a sampling command transmitted wirelessly, the microprocessor controls the sampling switch 24 to open the connecting tube 23 to start sampling. At the end of sampling, the microprocessor controls the sampling switch 24 to close the connecting tube 23 to prevent the sample from leaking or being contaminated by substances downstream of the gastrointestinal tract.

Further, as shown in FIG. 1 and especially FIG. 2, the sampling assembly 2 comprises a plurality of outer sampling ports 22 on the enclosure 1 and a sampling cavity 22′ connected to the plurality of outer sampling ports 22. The connecting tube 23 is connected to the sampling cavity 22′ and thus indirectly connected to the outer sampling ports 22. Gases and fluids in the gastrointestinal tract flow into the sampling cavity 22′ through the outer sampling ports 22 and converge into the connecting tube 23. Therefore, even when some of the outer sampling ports 22 are blocked, the rest remains unblocked and does not affect the sampling. In addition, the sampling cavity 22′ allows mixing and buffering of digestive fluids entering through each of the outer sampling ports 22 to ensure uniform and smooth sampling.

The plurality of outer sampling ports 22 are spaced along the circumference of the sampling capsule 100, and preferably equally spaced. In the embodiment, 3-5 said outer sampling ports 22 are cut in the enclosure 1, which can ensure smooth sampling without affecting the strength of the enclosure 1.

Preferably, the aperture diameter of each of the outer sampling ports 22 is smaller than that of the connecting tube 23, so that the substances that can pass through the outer sampling ports 22 into the sampling cavity 22′ cannot block the connecting tube 23.

Further, the sampling cavity 22′ comprises a filtering structure (not shown in FIGS.), such as, but not limited to, a filter screen, to avoid food residues blocking the connecting tube 23. For the filtering structure, the “An anti-blocking and anti-air suction structure in a digestive fluid sampling capsule 100” in Chinese Patent Application No. 201811330328.4 can be referenced.

In conjunction with any type of the sampling assembly 2, the mucosal flora collection auxiliary assembly 3 comprises a vibration motor 31 inside the enclosure 1 and/or a counterweight 32 at the outer sampling port 22.

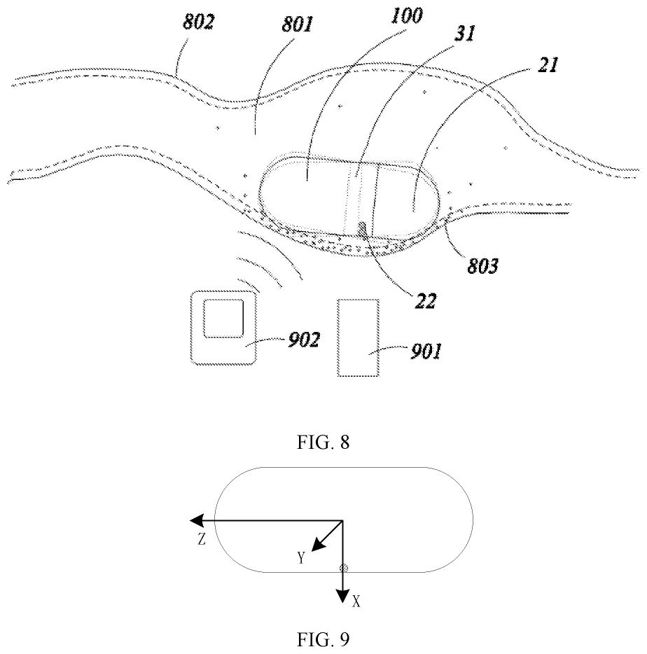

In general, when the sampling capsule 100 is in a region to be examined in the gastrointestinal tract or reaches a region having lesion in the gastrointestinal tract, sampling can be started. Upon arrival of the sampling capsule 100 at a region in the gastrointestinal tract where sampling is needed, the sampling capsule 100 is immersed into the digestive fluid under the action of the counterweight 32, and the outer sampling port 22 is directed toward and close to the wall of the gastrointestinal tract. Or, as shown in FIG. 8, the vibration motor 31 is started to vibrate the sampling capsule 100 and thereby stir the fluid in the region of the gastrointestinal tract, allowing the mucosal flora to enter the digestive fluid. Or while the counterweight 32 is operating simultaneously with the vibration motor 31, the connecting tube 23 is opened by the sampling switch 24, so that the digestive fluid with mucosal flora enters the sample chamber 21 through the outer sampling port 22 and the connecting tube 23 under the pressure difference between the internal and external environments. After sampling is completed, the connecting tube 23 can be closed by the sampling switch 24 to prevent the sample from leaking or being contaminated, and the sample volume can be precisely controlled by controlling the opening state and time of the connecting tube 23. In addition, the sampling switch 24 actively controls the opening or closing of the connecting tube 23 to make the sampling capsule 100 free to suck sample fluid in any region of the gastrointestinal tract without being affected by a special environment therein. Therefore, the sampling capsule has a high versatility.

The vibration motor 31, when it is turned on, can cause the sampling capsule 100 to vibrate in the gastrointestinal tract. In one aspect, vibration of the sampling capsule 100 can stir the surrounding fluid environment to make some of the colonies in the mucus layer enter the digestive fluid in the intestinal lumen. And for another, the vibrating of the sampling capsule 100 can more easily unfold the gastrointestinal tract, causing the sampling capsule 100 to get closer to the inner wall of the gastrointestinal tract and making the outer sampling port 22 contact the mucus layer.

Preferably, the vibration motor 31 is disposed in the central position of the sampling capsule 100, so that the sampling capsule 100 can be driven to vibrate more smoothly without deflection, and the vibration is easy to control. The “central position” herein is not a geometrical center of symmetry, but rather a small area centered on the center of symmetry, as long as it allows the sampling capsule 100 to vibrate smoothly. For example, the vibration motor 31 is disposed in the central position of the vibration motor 31 along the axis of the sampling capsule 100.

Preferably, the vibration amplitude of the vibration motor 31 is adjustable so that a suitable vibration amplitude can be used according to the specific situation, which is conducive to better collection of mucosal flora. Specifically, the vibration motor 31 communicates with the microprocessor. The microprocessor adjusts the output frequency and speed of the vibration motor 31 according to the commands sent from an external device to the sampling capsule 100, so as to adjust the vibration amplitude. Specifically, this can be achieved by adjusting the input voltage or current of a motor of the vibration motor 31. For example, a PWM (Pulse Width Modulation) technique can be used for such adjustment.

Specifically, the vibration motor 31 can be a button-type vibration motor, a coreless motor with an eccentric device, or a linear vibration motor. Preferably, a button-type vibration motor with smallest thickness and less space occupation is used. In addition, the direction of vibration of the vibration motor 31 is adjusted to be perpendicular to the outer sampling port 22.

Referring to FIG. 9, the direction of the outer sampling port 22 is defined as X direction, and the center of the sampling capsule 100 is set as the origin. When there is one outer sampling port 22, the direction of the origin pointing to the outer sampling port 22 is the X direction. When there are a plurality of outer sampling ports 22, with the outer sampling port 22 at the center as the endpoint, the direction of the origin pointing to the endpoint is the X direction. For example, when there are three outer sampling ports 22, the second outer sampling port is the endpoint; when there are four outer sampling ports 22, the midpoint of the middle two outer sampling ports is the endpoint. The direction of the long axis of the sampling capsule 100 is defined as the Z direction, and that both the Y and X directions are along the diameter of the sampling capsule 100, wherein the direction of vibration of the vibration motor 31 can be along the Y or Z direction, perpendicular to the X direction.

The counterweight 32 deflects the center of gravity of the sampling capsule 100 towards the outer sampling port 22. When the sampling capsule 100 is at a region where sampling is needed, the sampling capsule 100 is immersed into the digestive fluid under the action of the counterweight 32, so that the outer sampling port 22 faces and get close to the wall of the gastrointestinal tract, facilitating the collection of mucosal flora.

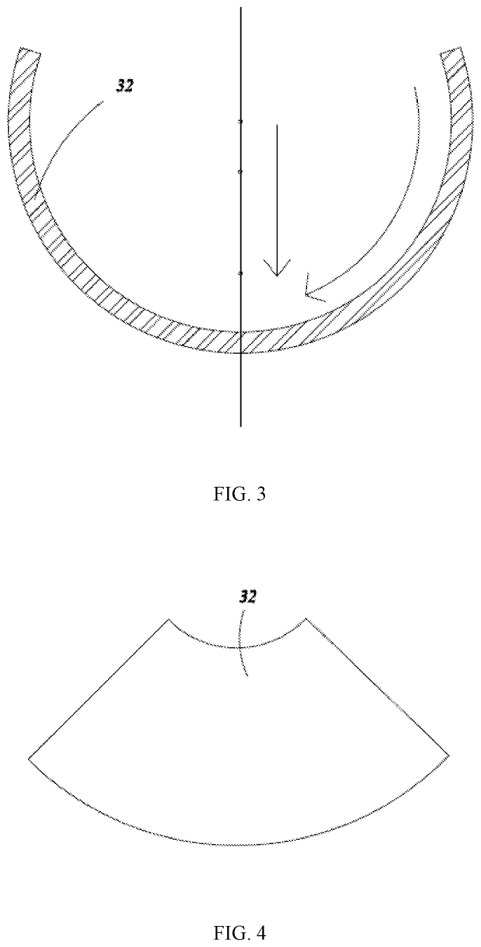

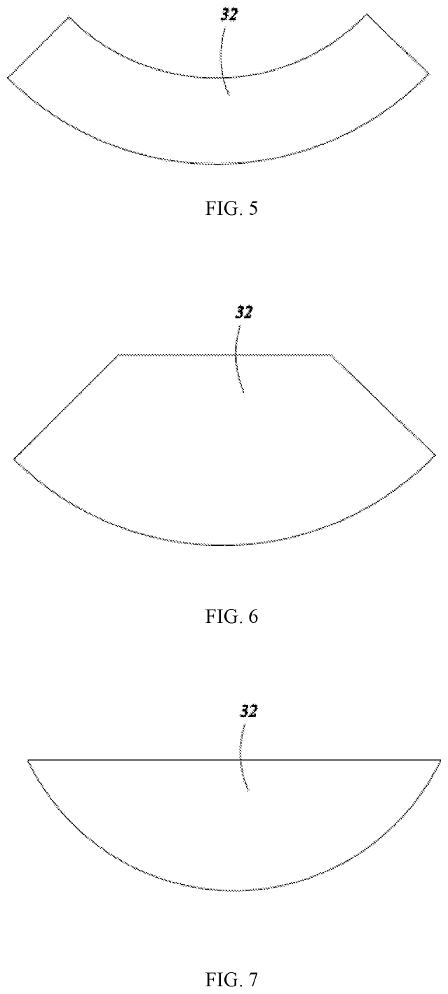

Preferably, as shown in FIGS. 3 to 7, the counterweight 32 is a magnetic member, and the center of gravity of the magnetic member is inclined to the side thereof close to the enclosure 1. The magnetic member can not only serve as a counterweight 32, and can also control the capsule movement or change the capsule posture of the sampling capsule 100 by an external magnetic field, so as to make the outer sampling port 22 be aligned with the wall of the gastrointestinal tract. When the sampling capsules 100 comprises the vibration motor 31, and the vibration motor 31 comprises a magnet, the magnet is magnetized in the same direction as the magnetic member as far as possible to avoid affecting the effect of the magnetic control.

Specifically, one side of the magnetic member is curved to match the enclosure 1. That is, the shape of one side of the magnetic member is consistent with that of the enclosure 1, and both are curved. Such structure allows the magnetic member to be mounted as close as possible to the enclosure 1 with the center of gravity of the magnetic member closer to the enclosure 1, and to be easily controlled by the external magnetic field.

Further, the magnetic member is radially magnetized, which is more conducive to the control by the external magnetic field.

For example, as shown in FIGS. 3 to 7, the magnetic member can be a radially magnetized NdFeB permanent magnet, and the shape of the magnetic member can be annular, fan-shaped, or partially ring-shaped.

When the magnetic member is fan-shaped or partially ring-shaped, its center of gravity is off the center of circle but towards the side of the outer sampling port 22, which acts as a counterweight, i.e., the center of gravity of the magnetic member is between the axis of the sampling capsule 100 and the outer sampling port 22. And, the shorter length the magnetic member has, the smaller an opening angle is and the closer the center of gravity is to the outer sampling port 22.

Preferably, the fan angle of the magnetic member is the same as the fan angle of the outer sampling port 22. The fan angle of the outer sampling port 22 refers to the angle of the fan formed by two ends of the outer sampling port 22 on the circumference of the sampling capsule 100 connecting the axis of the sampling capsule 100 along the diameter. When the sampling capsule 100 comprises a plurality of the outer sampling ports 22, the fan angle of the outer sampling ports 22 refers to the angle of the fan formed by the ends of the two outer sampling ports 22 on the circumference of the sampling capsule 100 connecting the axis of the sampling capsule 100 along the diameter.

For a partially ring-shaped structure, if the thickness and radius are the same, the larger the ring, the greater the opening angle, the closer the center of gravity to the center; the smaller the ring, the smaller the opening angle, the more the center of gravity deviates from the center of the circle. However, the smaller the opening angle, the smaller the size of the magnetic member, and correspondingly, its magnetic moment strength is reduced, and the magnetic control capacity is weakened. In addition, its weight is reduced, the impact on the distribution of the whole capsule center of gravity is also weakened, so it is necessary to select the opening angle with optimal magnetic control and counter weight effect according to the overall capsule weight and volume design.

The magnetic member is mounted in close proximity to the outer sampling port 22. Specifically, the magnetic member and the outer sampling port 22 are arranged side-by-side along the axis of the sampling capsule 100, so that the center of gravity of the sampling capsule 100 is inclined to the end of the outer sampling port 22 to act as a counterweight, and at the same time the capsule movement can be controlled or posture changed through the external magnetic field.

During sampling, the sampling capsule 100 can be controlled by the external magnetic field to get close to the intestinal wall and have the posture changed so that the outer sampling port 22 is facing the mucosa of the intestinal wall (see FIG. 2). In natural state, the outer sampling port 22 is more likely to face downward, submerged beneath the liquid surface. In this way, when the external magnetic field is difficult to affect the capsule, such as excessive distance that causes the magnetic force to be too weak, the outer sampling port 22 is still more easily immersed into the fluid, facilitating sampling.

In addition, the sampling capsule 100 further comprises a pressure sensor 5 disposed within the sample chamber 21 and the pressure sensor 42 detects pressure within the sample chamber 21. The control module 4 determines whether the sampling capsule 100 is valid based on the pressure before taking the sampling capsule 100. The control module 4 can also determine whether the sampling capsule 100 is valid based on the pressure before sending sampling commands. The control module 4 can also determine whether the sampling is proceeding properly, and determine whether the sampling ends.

Preferably, the pressure sensor 5 has an absolute pressure measurement range of 260 hPa to 1260 hPa, or has an absolute pressure measurement range of 300 hPa to 1100 hPa. If the 260 hPa 1260 hPa is selected, it can still display 260 hPa when the pressure is lower than 260 hPa. Therefore, after evacuating the sample chamber 21, the pressure value is observed and if it displays 260 hPa, it indicates that the absolute pressure is less than 260 hPa, i.e., 0 hPa˜260 hPa. If the 300˜1100 hPa is selected, when the actual pressure is lower than 300 hPa, the pressure sensor 5 can still give a measurement value, such as 80 hPa, but the measurement value is not accurate enough and it can only be judged qualitatively that the actual pressure is less than 300 hPa.

The sampling capsule 100 further comprises a temperature sensor 6 located in the enclosure 1, which has a temperature measurement range of 0° C. to 80° C., and is used for detecting the temperature at which the sampling capsule 100 is working to ensure that the sampling capsule 100 is working properly and that it cannot cause harm to the human body. When the temperature sensor 6 detects that the temperature exceeds a certain safety threshold, the sampling capsule needs to be shut down in time for cooling. The safety threshold is 55° C. to 60° C. so as not to cause harm to the human body.

Preferably, the temperature sensor 6 is disposed on the second side of the partition wall 13. For one thing, such arrangement does not contaminate the sample chamber 21. And for another, the element that generates heat when the sampling capsule 100 is working is substantially on the second side of the partition wall 13, so that the temperature of the sampling capsule 100 can be better detected.

The temperature sensor 6 is primarily used to monitor the temperature of the sampling switch 24. According to the different implementations of the sampling switch 24, such as shape memory miniature valve, motor-based valve, etc., heat can be generated during its operation.

The control module 4 further comprises a sensor 42 for collecting physiological parameters and/or image information in the gastrointestinal tract, and the sensor 42 communicates with the microprocessor. The sensor 42 can be one or more sensors selected from an image sensor, a pH sensor, or an ultrasonic sensor. When the sensor 42 comprises an image sensor, part of the enclosure 1 is transparent, and when the sensor 42 comprises a pH sensor, the enclosure 1 comprises a window. The specific method of determining which region of the gastrointestinal tract the sampling capsule 100 is in, based on the picture and pH value obtained by the sensor 42, can be any method in the prior art, and is not be repeated herein.

While the control module 4 comprises the sensor 42, the control module 4 can further comprise a storage module for storing normal physiological parameters or image information and physiological parameters or image information in case of possible lesions in different regions of the gastrointestinal tract, where the storage module communicates with the microprocessor. After the sensor 42 collects physiological parameters and/or image information in the gastrointestinal tract, the microprocessor compares the collected information with the stored information in the storage module to determine whether the sampling capsule 100 reaches the position at which the sample is to be taken.

Or, while the control module 4 comprises the sensor 42, the control module 4 further comprises a wireless transmission module for communicating with the external device. When the sensor 42 collects physiological parameters and/or image information in the gastrointestinal tract, it transmits the information to the external device, and the external device analyzes the information and determines whether the sampling capsule 100 reaches the position at which the sample is to be taken.

In addition, the control module 4 further comprises a battery that provides power to other components of the sampling capsule 100. The sampling capsule 100 further comprises a circuit board 41, and the microprocessor, the wireless transmission module, and the battery are all integrated on the same circuit board 41.

The process of using the sampling capsule 100 is described in detail below.

Referring to FIG. 8 in combination with FIGS. 1 to 7, taking collection of mucosal flora in the intestinal tract as an example. When the sampling capsule 100 reaches a designated intestinal lumen 801, an external magnet 901 can be used to approach the body from below so that the outer sampling port 22 is facing downward and getting close to the intestinal wall. Since the magnetic member is an annular radially magnetized magnet or a fan-shaped radially magnetized magnetic member disposed only on one side of the outer sampling port 22, the operator can control the rotation of the sampling capsule 100 along the capsule axis with reference to the image so that the end of the outer sampling port 22 is facing the intestinal wall 802. However, due to the folds of the intestinal wall 802, insufficient adsorption of the external magnet 901 caused by being too far away, and other reasons, the outer sampling port 22 may not be tightly attached to the mucus layer 803. A “vibration” command can be sent to the sampling capsule 100 via the external device. Upon receipt of the command, the vibration motor 31 can drive the whole sampling capsule 100 to vibrate at a high frequency, particularly in a direction perpendicular to the outer sampling port 22, keeping the outer sampling port 22 facing the intestinal wall 802. This method allows the sampling capsule 100 to be closer to the intestinal wall 802, while stirring the mucus layer 803 and fluids in the intestinal lumen 801 to make some flora in the mucus layer 803 enter the luminal fluid. After a period of time, sampling command can be sent through the external device 902, at which point the sampling capsule 100 can collect fluid containing a higher concentration of mucosal flora. During sampling, the gastrointestinal environment on the side of the outer sampling port 22 can always be determined by the images collected by the image sensor 42 to ensure that this side is filled with fluid at the time of sampling. In this way, the sampling success rate of the sampling capsule 100 and the collection concentration of the mucosal flora can be significantly improved by a combined control of the vibration motor 31, the magnetic member and external magnet 901, and a closed-loop control by the images obtained by the image sensor 42 as feedback.

In order to collect mucosal flora of higher concentration, it is also possible to use the vibration of the sampling capsule 100 alone without relying on the external magnet 901, to allow the sampling capsule 100 to automatically complete sampling. When the sampling capsule 100 reaches the region to be sampled, the vibration motor 31 starts vibration mode, the sampling capsule 100 stirs the surrounding intestinal environment and increasing the concentration of muscoal flora in the fluid in the lumen. After a period of time, the vibration motor 31 stops vibration, and when the sampling capsule 100 completely stops vibrating, under the effect of the counterweight of the magnetic chamber, the outer sampling port 22 is facing downwards and getting close to the intestinal wall 802, and immersed into the fluid. Then, sampling can be started. In this method, similar effect can also be achieved.

In addition, after the sampling capsule 100 is discharged, the methods of taking out the sampled digestive fluid comprises, but is not limited to, opening the connecting tube 23 by the sampling switch 24 to allow the fluid to flow out through the connecting tube 23 and the outer sampling port 22 for pathological analysis.

Alternatively, the sampling capsule 100 further comprises a sample drawing assembly 7 cooperating with the sample chamber 21 for evacuating and sample drawing. The sample drawing assembly 7 comprises a drawing port 71 on the enclosure 1 located on the first side of the partition wall 13 and connected to the sample chamber 21, a fixing member 72 corresponding to the drawing port 71, and a silicone plug 73 fitted in the fixing member 72. Specifically, the fixing member 72 is secured to the enclosure 1, and the silicone plug 73 has an interference fit to the fixing member 72. By the sample drawing assembly 7, a user can use a syringe or the like to penetrate the silicone plug 73 to pump the air out of the sample chamber 21 to form a vacuum or to take sample out of the sample chamber 21.

It can be understood by those of skill in the art that any of the sampling assemblies 2, any of the mucosal flora collection auxiliary assemblies 3, and any of the control modules 4 can all work together to form the sampling capsule 100.

The present invention further provides a sampling capsule system 100 comprising any kind of the sampling capsules 100 as described above and an external device in communication with the control module 4. Specifically, the control module 4 communicates with the said external device by any of the methods in the prior art. It is not be repeated herein.

The present invention further provides a control method based on the sampling capsule 100 described above, comprising the steps of: determining whether sampling is needed, and if the sampling is needed, the sampling switch 24 opens the connecting tube 23, and after sampling ends, the sampling switch 24 closes the connecting tube 23. To determine whether sampling is needed, any one of the methods as described above can be used, and details are not described herein again. Any one of the methods as described above can be used for the sampling switch 24 to open or close the connecting tube 23, and details are not described herein again.

In summary, the sampling capsule 100 of the present invention can assist the sampling assembly 2 to collect mucosal flora through the mucosal flora collection auxiliary assembly 3, can suck sample fluid into the sample chamber 21 by opening the connecting tube 23 by the sampling switch 24, and after sampling is completed, can close the connecting tube 23 by the sampling switch 24 to prevent the sample from leaking or being contaminated, and can precisely control the sample volume by controlling the opening state and time of the connecting tube 23. In addition, the sampling switch 24 actively controls the opening or closing of the connecting tube 23 to make the sampling capsule 100 free to suck sample fluid in any region of the gastrointestinal tract without being affected by a special environment therein. Therefore, the sampling capsule has a high versatility.

It should be understood that, although the specification is described in terms of embodiments, not every embodiment merely includes an independent technical solution. Those skilled in the art should have the specification as a whole, and the technical solutions in each embodiment may also be combined as appropriate to form other embodiments that can be understood by those skilled in the art.

The present invention by no means is limited to the preferred embodiments described above. On the contrary, many modifications and variations are possible within the scope of the appended claims.

What is claimed is:

1. A sampling capsule, comprising:
an enclosure;
a sampling assembly comprising a sample chamber disposed in the enclosure, an outer sampling port on the enclosure, a connecting tube connecting the outer sampling port and the sample chamber, and a sampling switch for opening or closing the connecting tube;
a mucosal flora collection auxiliary assembly comprising a vibration motor disposed in the enclosure, and/or a counterweight at the outer sampling port; and
a control module comprising a microprocessor in communication with the sampling switch and the vibration motor.

2. The sampling capsule of claim 1, wherein the sampling assembly comprises a plurality of outer sampling ports and a sampling cavity connected to the plurality of outer sampling ports, and the connecting tube is connected to the sampling cavity.

3. The sampling capsule of claim 2, wherein the plurality of outer sampling ports are distributed with certain spacing along the circumference of the enclosure.

4. The sampling capsule of claim 2, wherein the aperture diameter of each of the outer sampling ports is smaller than the inner diameter of the connecting tube.

5. The sampling capsule of claim 2, wherein the sampling cavity comprises a filtering structure.

6. The sampling capsule of claim 1, wherein the vibration motor is a button-type vibration motor, a coreless motor with an eccentric device, or a linear vibration motor.

7. The sampling capsule of claim 1, wherein the vibration motor is disposed in the central position of the sampling capsule.

8. The sampling capsule of claim 1, wherein the counterweight is a magnetic member and the center of gravity of the magnetic member is inclined to a side of the magnetic member close to the enclosure.

9. The sampling capsule of claim 8, wherein one side of the magnetic member is curved to match the enclosure, and the magnetic member is radially magnetized.

10. The sampling capsule of claim 8, wherein the magnetic member and the outer sampling port are arranged side-by-side along the axis of the sampling capsule.

11. The sampling capsule of claim 1, wherein the sampling capsule further comprises a sample drawing assembly, the sample drawing assembly comprising a drawing port on the enclosure and connected to the sample chamber, a fixing member corresponding to the drawing port, and a silicone plug fitted in the fixing member.

12. The sampling capsule of claim 1, wherein the control module further comprises a sensor for collecting physiological parameters and/or image information in gastrointestinal tract, and the sensor communicating with the microprocessor; or
wherein the control module further comprises a sensor for collecting physiological parameters and/or image information in the gastrointestinal tract, and a storage module for storing normal physiological parameters or image information and physiological parameters or image information in case of possible lesions in different regions of the gastrointestinal tract, wherein both the sensor and the storage module communicate with the microprocessor; or
wherein the control module further comprises a sensor for collecting physiological parameters and/or image information in the gastrointestinal tract and a wireless transmission module for communicating with an external device, wherein the sensor communicates with the microprocessor.

13. The sampling capsule of claim 12, wherein the sensor is one or more sensors selected from an image sensor, a pH sensor, or an ultrasonic sensor, wherein when the sensor comprises an image sensor, part of the enclosure is transparent, and when the sensor comprises a pH sensor, the enclosure comprises a window.

14. A sampling capsule system, comprising a sampling capsule and an external device, wherein the external device is in communication with a control module of the sampling capsule;
wherein the sampling capsule comprising:.
an enclosure;
a sampling assembly comprising a sample chamber disposed in the enclosure, an outer sampling port on the enclosure, a connecting tube connecting the outer sampling port and the sample chamber, and a sampling switch for opening or closing the connecting tube;
a mucosal flora collection auxiliary assembly comprising a vibration motor disposed in the enclosure, and/or a counterweight at the outer sampling port; and
the control module comprising a microprocessor in communication with the sampling switch and the vibration motor.
